(19) United States
(12) Patent Application Publication
(10) Pub. No.: US 2002/0198502 A1
(43) Pub. Date: Dec. 26, 2002

(54) Medical occlusion prevention apparatus and method

(76) Inventor: Luke Vohsing, Granville, OH (US)

Correspondence Address: WALKER & JOCKE, L.P.A., 231 SOUTH BROADWAY STREET, MEDINA, OH, 44256, US

(21) Appl. No.: 10/174,408

(22) Filed: Jun. 17, 2002

Related U.S. Application Data
(60) Provisional application No. 60/299,960, filed on Jun. 21, 2001.

(51) Int. Cl. A61M 31/00

(52) U.S. Cl. 604/270; 604/251; 604/537

(57) ABSTRACT

An occlusion prevention apparatus for a gastrostomy button system is provided. The apparatus includes an occlusion prevention tube comprised of a corrugated tube. The occlusion prevention apparatus may be in surrounding relation about a gastrostomy extension tube comprised of a flexible tube connecting a feeding bag adapter and a gastrostomy button adapter. The occlusion prevention apparatus may include a slit through the wall of the corrugated tube, which is operative to enable the flexible tube portion of the gastrostomy extension tube to slide into and out of an interior portion of the occlusion prevention tube. The occlusion prevention tube is operative to reduce the occurrences of blockages of nutrition in the gastrostomy extension tube while an individual is sleeping, and thereby reduce the number of audible warnings output by an alarm device monitoring the feeding process.

### CROSS REFERENCE TO RELATED APPLICATIONS

This application claims the benefit of U.S. Provisional Application Serial No. 60/299,960 filed Jun. 21, 2001.

### TECHNICAL FIELD

This invention relates to a medical occlusion prevention apparatus and method. Specifically, this invention relates to an apparatus and method that is operative to reduce the occurrence of occlusions in a gastrostomy feeding tube extension.

### BACKGROUND ART

External feedings are necessary for individuals who are unable to absorb sufficient nutrition through regular meals. External feedings may be supplied through an external feeding system which is directly connected to the stomach of an individual through a skin level device. A typical external feeding system 10 is shown in FIG. 1. With this system a supplemental nutrition may be supplied as a liquid, such as Peptamen®. The liquid nutrition 12 is poured in a feeding bag 14. A supply tube 16 connected to the feeding bag is directed through a pump 18 which is operative to urge the liquid nutrition 12 through the supply tube 16. A standard feeding bag typically includes an adapter 20 which is operative to connect with an individual's feeding tube. For individuals with a gastrostomy tube or button 22 in their stomach, a gastrostomy extension tube 24 may be used to connect the adapter 20 of the feeding bag to the gastrostomy button. Examples of gastrostomy buttons include the Bard® Button and the Kimberly Clark/Ballard Medical Products Mic-Key®. An example of a gastrostomy extension tube includes the Kimberly Clark/Ballard Medical Products Mic® extension.

Such gastrostomy extension tubes may include a flexible plastic tube 26 with two adapters 28, 30 on opposed ends of the flexible tube 26. A first adapter 28 of the gastrostomy extension tube may, for example, be adapted to cooperatively engage with an adapter 20 of a feeding bag 14. A second adapter 30 of the gastrostomy extension tube may be adapted to cooperatively engage with the gastrostomy button 22 mounted through the skin of the individual. Once the gastrostomy extension tube is mounted between the feeding bag and gastrostomy button, the feeding bag will be in fluid communication with the stomach of the individual.

The exemplary system may be used to supply nutrition to an individual while the individual is sleeping. Unfortunately, if the individual moves to a position which bends, kinks or otherwise forms an occlusion in the flexible tube 26 of the gastrostomy extension tube 24, the system will be prevented from supplying nutrition to the individual. To remedy the occurrence of an occlusion in the flexible tube 26, the pump 18 may include an alarm 32 that is operative to emit an audible warning which indicates that the liquid nutrition is being blocked. Such an audible sound is generally loud enough to wake the individual so that they may move to a new position which removes the occlusion in the flexible tube 26.

Unfortunately for individuals that turn their bodies periodically while sleeping, the audible warning may wake them up many times during the night. As a result, the alarm may prevent individuals from getting a sufficient amount of sleep at night. This, in turn, may lead to fatigue and other conditions related to poor sleep. Consequently, there exists a need for an apparatus and method that is operative to reduce the number of occlusions in a MIC extension 26 and thereby reduce the number of times an individual is awakened with an alarm device.

### DISCLOSURE OF INVENTION

It is an object of an exemplary form of the present invention to provide an apparatus and method that is operative to reduce the number of audible warnings that are emitted by an alarm due to the detection of a blockage in a gastrostomy extension tube.

It is a further object of an exemplary form of the present invention to provide an apparatus and method that is operative to minimize the occurrence of occlusions in medical tubes placed in fluid communication with an interior portion of a body of an individual.

It is a further object of an exemplary form of the present invention to provide an apparatus and method that is operative to minimize the occurrence of occlusions in feeding tubes.

It is a further object of an exemplary form of the present invention to provide an apparatus and method that is operative to minimize the occurrence of occlusions in gastrostomy extension tubes.

Further objects of an exemplary form of the present invention will be made apparent in the following Best Modes for Carrying Out the Invention and the appended claims.

The foregoing objects maybe accomplished in an exemplary embodiment of the invention by a medical occlusion prevention apparatus, which includes an occlusion prevention tube placed in surrounding relation about at least the flexible tube portion of a gastrostomy extension tube. In one exemplary embodiment, the occlusion prevention tube is corrugated to include a plurality of alternating annular ridges and grooves. In addition, the exemplary embodiment of the apparatus may include a slit through the wall of the occlusion prevention tube which extends in the longitudinal direction between each end of the occlusion prevention tube. The exemplary occlusion prevention tube may be placed around the gastrostomy extension tube by passing the gastrostomy extension tube through the slit in the wall of the occlusion prevention tube.

In an exemplary embodiment, the apparatus may be produced from a thermoplastic corrugated beverage straw which is cut along one side to form a longitudinal slit in the wall of the straw. In this described exemplary embodiment, the straw may include a plurality of uniformly spaced ridges and grooves which are operative to minimize the formation of kinks or bends in the straw which could cause an occlusion in the gastrostomy extension tube.

In an exemplary embodiment, the occlusion prevention tube may have an inner diameter which is greater than the outer diameter of the flexible tube portion of the gastrostomy extension tube. In addition, the occlusion prevention tube may have a length less than or equal to the length of the gastrostomy extension tube. However, it is to be understood that in alternative exemplary embodiments, other inner and outer diameters of the occlusion prevention tube may be used based on the size of the gastrostomy extension tube to be protected, and the flexibility, strength, size and occlusion prevention characteristics desired for the occlusion prevention tube. For example, in an alternative exemplary embodiment, the occlusion prevention tube may have an inner diameter which is sufficient in size to surround the feeding bag adapter portion of the gastrostomy extension tube. As a result, the occlusion prevention tube may have a length which extends in surrounding relation about both the flexible tube portion of the gastrostomy extension tube and at least a portion of the feeding bag tube.

### BRIEF DESCRIPTION OF DRAWINGS

FIG. 1 is a schematic view of a feeding system for a gastrostomy button system.

FIG. 2 is a side plan view of an exemplary embodiment of an occlusion prevention apparatus.

FIG. 3 is a cross-sectional side view of a corrugated wall of the exemplary embodiment of the occlusion prevention tube.

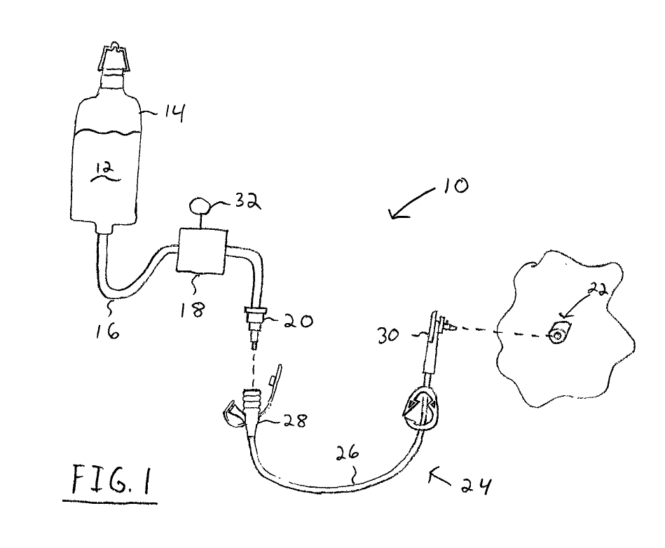

### BEST MODES FOR CARRYING OUT INVENTION

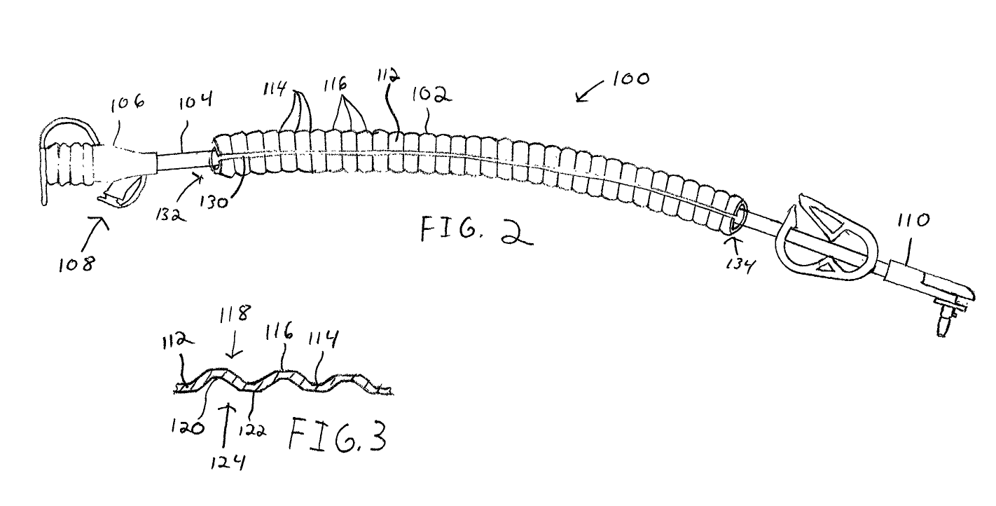

Referring now to the drawings and particularly to FIG. 2, there is shown therein a perspective view of an occlusion prevention apparatus 100. Here the apparatus 100 includes an occlusion prevention tube 102. The occlusion prevention tube is adapted to be placed in surrounding relation about a gastrostomy extension tube 108, such as a Kimberly Clark/Ballard Medical Products Mic® extension or other extension tube for a gastrostomy button system. In this described exemplary embodiment, the gastrostomy extension tube may include a feeding bag adapter portion 106 on one end of a flexible tube 104. The gastrostomy extension tube 108 may also include a gastrostomy button adapter 110 on an opposed end of the flexible tube 104. In the exemplary embodiment, the feeding bag adapter 106 may be operative to cooperatively connect the gastrostomy extension tube 108 to the adapter of a feeding bag to form a fluid tight seal with the feeding bag. Further, the gastrostomy button adapter 110 may be operative to connect the gastrostomy extension tube 108 to a gastrostomy button mounted to an individual's stomach to form a fluid tight seal with the stomach through the skin of the individual. Examples of gastrostomy buttons include the Bards® Button and the Kimberly Clark/Ballard Medical Products Mic-Key®.

As shown in FIG. 3, the exemplary embodiment of the occlusion prevention tube 102 includes a corrugated wall 112 comprised of a series of annular grooves 114 and ridges 116 along an outer surface 118 of the wall. The occlusion prevention tube 102 may further include a corresponding series of annular grooves 120 and ridges 122 along an inner surface 124 of the wall. The corrugated wall is operative to resist kinks and bends in the occlusion prevention tube 102 which may collapse the flexible tube portion 104 of the gastrostomy extension tube and block the flow of nutrition through the gastrostomy extension tube. For example, in an exemplary embodiment, the occlusion prevention tube may be comprised of a thermoplastic corrugated beverage straw. In alternative exemplary embodiments, the occlusion prevention tube may be made out of other flexible materials which can be formed into a corrugated tube including clear, semi-clear and opaque plastics, for example.

In an exemplary embodiment, inner ridges 122 of the occlusion prevention tube may have an inner diameter greater than the outer diameter of the extension tube 104. Also, the occlusion prevention tube may have a length which is about equal to, or is less than, the length of the flexible tube 104 between the feeding bag adapter 106 and the gastrostomy button adapter 110 of the gastrostomy extension tube 108. In an exemplary embodiment, the inner diameter of the inner ridges 122 may be at least about 0.5 centimeters, the outer diameter of the outer ridges 116 may be about 1 centimeter, and the length of the occlusion prevention tube may be about 26 centimeters. In this described exemplary embodiment, the outer and inner ridges 116, 122 may be equidistantly spaced about every 0.4 centimeters. However, it is to be understood that in alternative exemplary embodiments, other dimensions of the corrugated tube may be provided in the occlusion prevention apparatus based on the dimensions of the flexible tube 104 of the gastrostomy extension tube and the flexibility, size and strength of the apparatus desired.

As shown in FIG. 2, the exemplary form of the occlusion prevention tube may include a longitudinal slit 130 through the wall 112 that extends between opposed ends 132, 134 of the occlusion prevention tube 102. In the exemplary embodiment, the slit 130 may enable the flexible tube portion of the gastrostomy extension tube to be moved into and out of the interior portion of the occlusion prevention tube by sliding the flexible tube 104 through the slit 130. In an exemplary embodiment, the wall 112 of the occlusion prevention tube may be sufficiently flexible to enable the slit to be spread apart with the fingers of an individual. In addition, the wall 112 of the occlusion prevention tube may be sufficiently resilient to close, or at least narrow, the width of the slit once the flexible tube portion 104 has been passed through the slit. In the exemplary embodiment, the wall 112 resiliently returns to an orientation in which the slit is closed or returns to a width that is less than the width of the flexible tube portion 104 of the gastrostomy extension tube.

An exemplary method of using the occlusion prevention tube in a feeding system may include providing a feeding bag which is operative to store a liquid nutrition. A gastrostomy extension tube may be provided to place the feeding bag in fluid communication with the gastrostomy button connected to an individual's stomach. The occlusion prevention tube may be placed in surrounding relation about the gastrostomy extension tube by sliding the flexible extension tube portion of the gastrostomy extension tube through a slot in the corrugated wall of the occlusion prevention tube. In alternative exemplary embodiments, rather than sliding the gastrostomy extension through a slot, the inner diameter of the occlusion prevention tube may be sufficiently large to enable at least one end of the gastrostomy extension to slide through the occlusion prevention tube between opposed ends of the occlusion prevention tube.

In an exemplary method, the gastrostomy extension tube may be connected to the feeding bag by inserting a male adapter end of the feeding bag into a corresponding female feeding bag adapter of the gastrostomy extension tube. The gastrostomy button adapter of the gastrostomy extension tube may be operatively connected and locked into the gastrostomy button of an individual's stomach. After the feeding bag is filled with a liquid nutrition, a pump in operative connection with the feeding bag may be used to move nutrition through the feeding system from the feeding bag through the gastrostomy extension tube and into the stomach of the individual. In exemplary embodiments of the occlusion prevention tube which include a slit, an adhesive tape may be placed across the slit to prevent the gastrostomy extension tube from sliding back through the slit of the occlusion prevention tube.

Although the described exemplary forms of the apparatus have been described for use with a gastrostomy extension tube, in alternative exemplary embodiments, the occlusion prevention apparatus may be used to prevent occlusions in other types of medical tubes for directing nutrition, medicines, bodily fluids and other types of fluids to and from the interior portions of bodies of individuals.

For example, with feeding tubes placed through the nose or mouth of a patient, an alternative exemplary embodiment may include the method of placing a corrugated tube in surrounding relation about the feeding tube. Such a corrugated tube may be placed around the portion of the feeding tube that extends between the patient's mouth or nose and the source of the liquid nutrition. In general, the occlusion prevention tube may be positioned along the feeding tube adjacent portions of the feeding tube which have a possibility of being pinched, compressed or bent by the movement of the patient's body. As a result, if a patient accidentally rolls onto the feeding tube, or places a portion of their body on top of the feeding tube, the occlusion prevention tube is operative to prevent an occlusion from forming in the feeding tube.

Further alternative exemplary embodiments may include placing a corrugated tube around other medical tubes, such as tubes directing medications, plasma or other fluids into the veins of a patient. In addition, other alternative exemplary embodiments may include placing a corrugated tube around medical tubes directing urine, blood or other fluids away from the body of a patient.

Thus, the exemplary form of the new medical occlusion prevention apparatus and method achieves one or more of the above stated objectives, eliminates difficulties encountered in the use of prior devices and systems, solves problems and attains the desirable results described herein.

In the foregoing description, certain terms have been used for brevity, clarity and understanding, however no unnecessary limitations are to be implied therefrom, because such terms are used for descriptive purposes and are intended to be broadly construed. Moreover, the descriptions and illustrations herein are by way of examples and the invention is not limited to the exact details shown and described.

In the following claims, any feature described as a means for performing a function shall be construed as encompassing any means known to those skilled in the art to be capable of performing the recited function, and shall not be limited to the features and structures shown herein or mere equivalents thereof. The description of the exemplary embodiment included in the Abstract included herewith, shall not be deemed to limit the invention to features described therein.

Having described the features, discoveries and principles of the invention, the manner in which it is constructed and operated, and the advantages and useful results attained, the new and useful structures, devices, elements, arrangements, parts, combinations, systems, equipment, operations, methods and relationships are set forth in the appended claims.

I claim:

1. A medical occlusion prevention apparatus comprising:
a gastrostomy extension tube, wherein the gastrostomy extension tube includes:
a gastrostomy button adapter, wherein the gastrostomy button adapter is adapted to cooperatively form a fluid tight connection with a gastrostomy button;
a feeding bag adapter, wherein the fluid bag adapter is adapted to cooperatively form a fluid tight connection with a feeding bag;
a flexible tube between the gastrostomy button adapter and the feeding bag adapter; and
an occlusion prevention tube in surrounding relation about the flexible extension tube, wherein the occlusion prevention tube includes a corrugated wall, and wherein the corrugated wall includes a longitudinal slit therethrough that extends between opposed ends of the occlusion prevention tube.

2. The apparatus according to claim 1, wherein the occlusion prevention tube includes annular grooves and ridges along the outer and inner surfaces of the corrugated wall.

3. The apparatus according to claim 2, wherein the occlusion prevention tube includes a plurality of annular inner ridges with an inner diameter greater than the outer diameter of the flexible tube.

4. The apparatus according to claim 3, wherein the inner diameter of the plurality of annular inner ridges is at least about 0.5 centimeters.

5. The apparatus according to claim 4, wherein the occlusion prevention tube includes a plurality of outer annular ridges with an outer diameter of about 1 centimeter.

6. The apparatus according to claim 5, wherein the plurality of outer ridges are equidistantly spaced about very 0.4 centimeters.

7. The apparatus according to claim 1, wherein the gastrostomy extension tube includes a first end and a second end, wherein the first end includes the feeding bag adapter, wherein the second end includes the gastrostomy button adapter, wherein the occlusion prevention tube has a length that is generally less than the length of the flexible extension tube between the feeding bag adapter and the gastrostomy button adapter.

8. The apparatus according to claim 1, wherein the occlusions prevention tube is comprised of a thermoplastic corrugated straw.

9. The apparatus according to claim 1, wherein the occlusions prevention tube is sufficiently flexible to enable human hands to spread apart the slit to a width that is sufficient in size to enable the flexible tube of the gastrostomy extension tube to slide therethrough, wherein the occlusion prevention tube is sufficiently resilient to return to a position which prevents the flexible tube of the gastrostomy extension tube from sliding through the slit.

10. A method comprising:
a) providing a feeding bag;
b) providing a gastrostomy extension tube;
c) placing an occlusion prevention tube in surrounding relation about at least a portion of the gastrostomy extension tube;
d) placing a first end of the gastrostomy extension tube in fluid communication with the feeding bag;
e) placing a second end of the gastrostomy extension tube in fluid communication with a gastrostomy button connected to a stomach of an individual;
f) filling the feeding bag with a nutrition; and
g) pumping the nutrition from the feeding bag to the stomach of the individual through the gastrostomy extension tube.

11. The method according to claim 10, wherein in step (c) the occlusion prevention tube includes a corrugated wall with a longitudinal slit that extends between opposed ends of the occlusion prevention tube, wherein step (c) includes sliding the a flexible tube portion of the gastrostomy extension tube through the longitudinal slit and into an interior portion of the occlusion prevention tube.

12. The method according to claim 11, wherein step (c) includes spreading the slit apart.

13. The method according to claim 11, where after step (c) further comprising:
h) placing an adhesive tape across the slit of the corrugated tube in a position which is operative to prevent at least a portion of the flexible tube portion of the gastrostomy extension tube from sliding back through the slot.

14. A method comprising:
a) providing a flexible medical tube;
b) providing a corrugated tube, wherein the corrugated tube includes a longitudinal slit between opposed ends of the corrugated tube
c) spreading the slit apart;
d) sliding the flexible medical tube through the slit to place at least a portion of the medical tube within an interior portion of the corrugated tube;
e) enabling the corrugated tube to resiliently return to an orientation in which the flexible medical tube is unable to slide back through the slit;
f) placing the flexible medical tube in fluid connection with an interior portion of a body of an individual; and
g) directing fluids to move through the medical tube between an external reservoir in operative connection with the flexible medical tube and the interior portion of the body of the individual.

15. The method according to claim 14, wherein step (d) includes pumping a nutrition through the medical tube with a pump from the external reservoir to the interior portion of the body of the individual; and further comprising:
h) monitoring for blockages of the nutrition in the medical tube.

16. The method according to claim 14, wherein in step (g) the fluid includes food.

17. The method according to claim 14, wherein in step (g) the fluid includes medication.

18. The method according to claim 14, wherein in step (g) the fluid includes blood.

19. The method according to claim 14, wherein in step (g) the fluid includes urine.

20. The method according to claim 14, wherein in step (a) the flexible medical tube includes a gastrostomy extension tube; wherein step (f) includes connecting the gastrostomy extension tube to a gastrostomy button; wherein in step (g) the external reservoir includes a feeding bag.
